(19) United States
(12) Patent Application Publication
(10) Pub. No.: US 2018/0369327 A1
(43) Pub. Date: Dec. 27, 2018

(54) METHOD FOR TREATING OR PREVENTING OSTEOARTHRITIS

(71) Applicant: Dong-A University Research Foundation for Industry-Academy Cooperation, Busan (KR)

(72) Inventors: Young-Hyun YOO, Busan (KR); Sung-Won LEE, Busan (KR); Jee-Hyun RHO, Busan (KR)

(21) Appl. No.: 15/632,566

(22) Filed: Jun. 26, 2017

(51) Int. Cl. A61K 38/17; A61K 9/00; A61K 31/713; A61K 38/18

(52) U.S. Cl. CPC A61K 38/1703; A61K 9/0019; A61K 48/00; A61K 38/1875; A61K 31/713

(57) ABSTRACT

A method for treating or preventing osteoarthritis includes promoting the expression of STAMP2 in chondrocyte. Osteoarthritis is diagnosed by measuring an expression level of STAMP2 or an amount of STAMP2 protein in chondrocyte, and comparing the measured results with that in a control sample. A therapeutic agent for osteoarthritis is screened by treating chondrocyte with a candidate agent, measuring an expression level of STAMP2 or an amount of STAMP2 protein in the chondrocyte, and identifying a candidate agent as the therapeutic agent when the measured expression level of STAMP2 or the measured amount of STAMP2 protein is increased in the chondrocyte as compared to the chondrocyte before treatment with the candidate agent. Measuring the expression level of STAMP2 or the amount of extracellularly secreted STAMP2 protein is highly useful for diagnosing osteoarthritis in a rapid and convenient manner using a biological sample such as blood.

### BACKGROUND

### 1. Field of the Invention

The present invention relates to a method for treating or preventing osteoarthritis (OA).

### 2. Discussion of Related Art

Osteoarthritis (OA) is a multifactorial disease characterized by degradation of the extracellular matrix and destruction of articular cartilage. Because chondrocytes are the only resident cells in human articular cartilage, on which cells matrix turn over in cartilage is solely dependent. Thus, the death of articular chondrocytes is generally considered to plays a central role in OA cartilage destruction. To date, stimuli involved in chondrocyte death and their signaling pathways have been in the spotlight as pathogenetic factors leading to joint cartilage degradation.

OA is now considered more complex disease with different clinical subtypes. Among these subtypes, metabolic OA is distinguished from different subtypes by the presence of obesity or metabolic syndrome and low-grade systemic inflammation and the earlier onset and a faster progression. The concept of “wear and tear disease”, which is traditionally accepted for the pathophysiology of OA does not seem to account for the cartilage destruction in metabolic OA. Not only joint overload is unable to explain strong epidemiological data which have shown the association between obesity and hand OA, but obese patients with metabolic syndrome have increased risk of knee OA compared to obese patients without metabolic syndrome. Thus, systemic factors must be involved in the pathogenesis of OA. Recent studies led to the discovery of proinflammatory cytokines and adipokines produced by the adipose tissue as central contributors of metabolic OA of the hand but potentially other locations.

Lipid imbalance is a key metabolic alteration associated with metabolic syndrome and obesity. In hyperlipidemic states, lipids abnormally accumulate in no-adipose tissues. Articular chondrocytes unlike most other cells is characterized as having substantial stores of lipid deposits. A previous study demonstrated the existence of a marked and graded increase in total fatty acids in articular cartilage from osteoarthritic joints. In hyperlipidemic states, accumulation of excess lipid in non-adipose tissue, exerts lipotoxicity, leading to cell dysfunction and/or cell death. Free fatty acids (FFAs), which are elevated associated with metabolic syndrome or obesity, have been considering the principal offender exerting lipotoxicity, inducing the apoptosis, the insulin resistance and inflammation. Thus, it would have been readily presumable that the accumulation of FFA contributes to OA pathogenesis. However, the causal relationship between FFA and OA pathogenesis was recently demonstrated. A pioneering study demonstrated that palmitate but not oleate has proapoptotic effect on interleukin 1 beta (IL-1-β)-stimulated articular chondrocytes, suggesting that elevated levels of saturated FFA may contribute to OA pathogenesis.

### SUMMARY

The present invention is directed to providing a method for treating or preventing osteoarthritis in a subject, comprising promoting the expression of STAMP2 (Six-transmembrane protein of prostate 2) in chondrocyte in chondrocyte in the subject.

Also, the present invention is directed to providing a method for diagnosing osteoarthritis measuring the expression level of STAMP2 or the amount of STAMP2 protein.

Also, the present invention is directed to providing a method for screening a therapeutic agent for osteoarthritis.

However, the problems to be solved according to the present invention are not limited to the above-described problems, and other problems which are not disclosed herein may be made apparent to those skilled in the art by the detailed description provided below.

This invention discloses a method for treating or preventing osteoarthritis in a subject, comprising promoting the expression of STAMP2 (Six-transmembrane protein of prostate 2) in chondrocyte in chondrocyte in the subject.

In some embodiments of the present invention, the promoting the expression of STAMP2 is by administering cilostazol or TNF-α, to a subject.

In some embodiments of the present invention, the administration is an oral or intravenous administration.

In some embodiments of the present invention, the promoting the expression of STAMP2 is by administering a vehicle, into which a gene encoding STAMP2 is introduced, to a subject.

In some embodiments of the present invention, the vehicle is an adenovirus.

In some embodiments of the present invention, the administration is intravenous administration.

This invention discloses a method for diagnosing osteoarthritis, comprising following steps: a) measuring an expression level of STAMP2 or an amount of STAMP2 protein in chondrocyte from the subject; and b) comparing the measured results in step a) with that in a control sample.

In some embodiments of the present invention, the expression level of STAMP2 is measured by any one selected from the group consisting of RT-PCR, Competitive RT-PCR, Realtime RT-PCR, RPA (RNaseprotection assay), Northern blotting and DNA chip.

In some embodiments of the present invention, the amount of STAMP2 protein is measured by any one selected from the group consisting of Western blot, ELISA (enzyme linked immunosorbent assay), RIA (radioimmunoassay), radioimmunodiffusion, Ouchterlony immunodiffusion, rocket immunoelectrophoresis, INC. (immunohistochemistry), Immunoprecipitation assay, complement fixation assay, FACS (fluorescence activated cell sorter) and protein chip.

This invention discloses a method for screening a therapeutic agent for osteoarthritis, comprising following steps: a) treating chondrocyte with a candidate agent; b) measuring an expression level of STAMP2 or an amount of STAMP2 protein in the chondrocyte treated with the candidate agent; and c) identifying a candidate agent as the therapeutic agent when the expression level of STAMP2 or the amount of STAMP2 protein measured in step b) is increased in the chondrocyte as compared to the chondrocyte before treatment with the candidate agent.

### BRIEF DESCRIPTION OF THE DRAWINGS

FIGS. 1A and 1B. HFD accelerates the onset of OA. The onset of OA was determined by the irregular surface and the disappearance of surface layer cells from tissues stained with H&E and reduced Safranin O. Scale bars, 50 μm. (A) Mice fed a HFD or a SD for 12 weeks were subjected to surgery for experimental OA, and after 4 weeks (n=10 for each diet), 6 weeks (n=10 for each diet) and 8 weeks (n=10 for each diet), the cartilage was observed. Representative histologic findings show that characteristic OA findings were observed 6 weeks after surgery in mice fed a HFD but not in mice fed a SD. The graph shows the occurrence of the onset of OA among mice fed a HFD and a SD during three different weeks. The dietary variable showed a significant difference between HFD and SD groups based on the likelihood ratio test (P<0.05) from Poisson regression analysis. (B) Mice were fed a HFD or a SD for 25 weeks, and the cartilage was observed. Characteristic OA findings were observed in all mice fed a HFD (n=10) but not in mice fed a SD (n=10). The graph shows the occurrences of the onset of OA among 10 mice for the HFD and SD groups. There was a significant difference between HFD and SD feeding (P<0.01) according to the chi-squared test.

FIGS. 2A to 2E. FFAs exert lipotoxicity in rat articular chondrocytes. (A) Viability assay. Palmitate (P) or stearate (S) at 0.5 to 2 mM and oleate (O) at 1.5 to 2.0 mM significantly reduced cell viability (n=4). ** P<0.01 versus vehicle according to the Kruskal-Wallis nonparametric test. (B) Representative histograms showing cell cycle progression and the induction of apoptosis (Apo, the percentage of the population undergoing apoptosis). Oleate at 1.5 mM markedly increased the number of apoptotic cells. (C) Representative Hoechst staining. The quantification of staining demonstrates that 1.5 mM oleate significantly increased the number of cells with condensed apoptotic nuclei. P<0.01 versus vehicle according to the Kruskal-Wallis nonparametric test. Scale bar, 20 μm. (D) Representative Western blots showing that 1.5 mM oleate significantly induced the activation of apoptosis-related factors (n=4). (E) Viability assay showing that zVAD-fmk significantly prevented 1.5 mM oleate-induced cell death (n=4). ** P<0.01 according to the Kruskal-Wallis nonparametric test.

FIGS. 3A to 3C. LD accumulation confers resistance in articular chondrocytes to oleate-caused lipotoxicity. (A) Representative Nile red and BODIPY staining. While oleate at 1-1.25 mM induced giant LD, the LD size was much smaller in cells treated with 1.5 mM oleate. The quantification of the staining showed that 1.5 mM oleate reduced the total LD volume (n=4). ** P<0.01 versus 1 mM oleate treatment according to the Kruskal-Wallis nonparametric test. Scale bars, 20 μm. (B) Representative Western blots showing that 1.5 mM oleate decreased the expression level of FSP27 protein (n=4). (C) Representative TUNEL, Hoechst and BODIPY triple-labelling. Scale bars, 20 μm. The size of the LDs decreased in TUNEL-positive cells. The quantification of the staining showed that the total LD volume was significantly decreased in TUNEL-positive cells treated with 1.5 mM oleate. ** P<0.01 versus TUNEL(−) cells treated with 1 mM oleate according to the Kruskal-Wallis nonparametric test. Scale bars, 20 μm.

FIGS. 4A to 4H. PKCK2 inhibition prohibits oleate-induced LD accumulation through FSP27. (A) Viability assay showing that FFAs sensitized chondrocytes to 100 μM DRB-induced death (n=4). * P<0.05 and ** P<0.01 versus FFA alone treatment according to the Kruskal-Wallis nonparametric test. (B) Representative Western blots showing that FFAs at toxic concentrations reduced the expression level of PKCK2 protein (n=4). (C) Representative Western blots showing that 1 mM oleate with 100 μM DRB decreased the levels of FSP27 protein and activated caspase-7 and -3 (n=4). (D) Representative confocal microscopy images showing that co-treatment with DRB reversed oleate-induced LD enlargement. The quantification shows that the total LD volume was significantly decreased by co-treatment with DRB (n=4). ** P<0.01 versus 1 mM oleate treatment according to the Kruskal-Wallis nonparametric test. Scale bar, 10 μm. (E) Viability assay showing that cilostazol (Cilo) pre-treatment prevented 1.5 mM oleate-induced cell death (n=4). * P<0.05 and ** P<0.01 versus OA alone treatment according to the Kruskal-Wallis nonparametric test. (F) Representative histograms showing that 30 μM cilostazol prevented 1.5 mM oleate-induced apoptosis (n=4). (G) Representative Western blots showing that 30 μM cilostazol prevented the 1.5 mM oleate-induced downregulation of FSP27 and the activation of caspase-3 and -7 (n=4). (H) Representative confocal microscopy images showing that cilostazol prevented oleate-induced LD fragmentation (n=4). Quantification shows that the total LD volume was significantly increased by cilostazol. ** P<0.01 versus 1.5 mM oleate alone treatment according to the Kruskal-Wallis nonparametric test. Scale bar, 10 μm.

FIGS. 5A to 5E. STAMP2 confers resistance to articular chondrocytes against oleate-induced lipotoxicity, and the population of STAMP2-positive cells is decreased in mice fed a HFD. (A) Representative Western blots showing that STAMP2 was substantially expressed in rat articular chondrocytes (Chon) (n=4). Liver, adipose tissue (Adip) and heart were used as positive controls. (B) Representative Western blots showing that oleate (O), palmitate (P) or stearate (S) at toxic concentrations reduced the expression level of STAMP2 (n=4). (C) Viability assay showing that STAMP2- or FSP27-depletion significantly sensitized chondrocytes to lipotoxicity (n=4). ** P<0.01 versus scRNA according to the Kruskal-Wallis test. (D) The knockdown of STAMP2 or FSP27 significantly reduced the total LD volume. ** P<0.01 versus scRNA control according to the Kruskal-Wallis nonparametric test. Scale bar, 10 μm. (E) Immunohistochemistry staining of the cartilage obtained from experimental OA mice subjected to surgery shows that the population of STAMP2-, PKCK2- or FSP27-positive articular chondrocytes 6 weeks after surgery was significantly smaller in mice fed a HFD than in mice fed a SD. The STAMP2-, PKCK2- or FSP27-positive cells were quantified (n=10). ** P<0.01 versus mice fed a SD according to the Kruskal-Wallis nonparametric test. Scale bar, 50 μm.

FIGS. 6A to 6F. PKCK2/STAMP2/FSP27-mediated LD accumulation rescues dietary fat-associated OA chondrocytes. (A) Representative Western blots showing that DRB decreased the expression level of STAMP2 and FSP27 (n=4). (B) Representative Western blots showing that 30 μM cilostazol (Cilo) reversed the 1.5 mM oleate (O)-induced downregulation of STAMP2 (n=4). (C) Representative immunohistochemistry showing that cilostazol markedly prevented the HFD-induced cartilage destruction and that cilostazol significantly increased the populations of PKCK2, STAMP2 or FSP27-positive cells (n=10). ** P<0.01 versus mice fed SD according to the Kruskal-Wallis nonparametric test. Scale bar, 50 μm. (D) Representative Western blots showing that STAMP2 was efficiently overexpressed by Ad-STAMP2 in articular chondrocytes. Viability assay showing that overexpressed STAMP2 significantly prevented oleate-induced cell death. ** P<0.01 versus 1.5 mM oleate alone treatment according to the Kruskal-Wallis nonparametric test. (E) Representative Western blots showing that overexpressed STAMP2 (1,000 MOI) reversed the 1.5 mM oleate-induced decrease in FSP27 and PKCK2 protein expression levels (n=4). (F) The overexpression of STAMP2 (1,000 MOI) reversed the 1.5 mM oleate-induced reduction in the total LD volume. ** P<0.01 versus empty vector (Ad-EV) according to the Kruskal-Wallis nonparametric test. Scale bar, 10 μm.

FIGS. 7A to 7E. Articular chondrocytes co-incubated with palmitate and oleate also survive PKCK2/STAMP2/FSP27-mediated LD accumulation. (A) A viability assay showing that 0.2 and 0.4 mM oleate (O) supplementation suppressed palmitate (P)-induced lipotoxicity (n=4). * P<0.05 and ** P<0.01 versus experimental control (0.4-1.8 mM palmitate alone treatment) according to the Kruskal-Wallis nonparametric test. (B) A viability assay showing that 0.4 mM oleate significantly reversed 0.6 and 1.6 mM palmitate-induced lipotoxicity and that PKCK2 inhibition by DRB and PKCK2 augmentation by cilostazol showed reciprocal influences on lipotoxicity (n=4). ** P<0.01 versus experimental control according to the Kruskal-Wallis nonparametric test. (C) LD accumulation was associated with resistance to the lipotoxicity induced by the combination treatments (n=4). * P<0.05 and ** P<0.01 versus scRNA control according to the Kruskal-Wallis nonparametric test. Scale bar, 10 μm. (D) Representative Western blots showing that the increase in the LD accumulation in chondrocytes co-treated with 0.4 mM oleate and 0.6 mM palmitate or 0.4 mM oleate and 1.6 mM palmitate was correlated with the increase in the expression level of PKCK2, STAMP2 and FSP27 (n=4). (E) A viability assay showing that siSTAMP2 and siFSP27 significantly decreased the viability of chondrocytes co-treated with 0.4 mM oleate and 0.6 mM palmitate (n=4). ** P<0.01 versus experimental control according to the Kruskal-Wallis nonparametric test.

FIGS. 8A to 8D. The total cytosolic FFA content is higher in articular chondrocytes that succumb to FFA-induced lipotoxicity than in articular chondrocytes that survive lipotoxicity. (A) The FFA level in the cytosol of articular chondrocytes treated with 1.25 mM or 1.5 mM oleate (O). 1.5 mM oleate significantly increased FFA level in the cytosol compared to experimental control. ** P<0.01 versus experimental control according to the Kruskal-Wallis nonparametric test. (B) The FFA level in the cytosol of articular chondrocytes treated with 1.0 mM oleate with or without DRB. DRB significantly increased the FFA level in the cytosol compared with that in the experimental control. ** P<0.01 versus experimental control according to the Kruskal-Wallis nonparametric test. (C) The FFA level in the cytosol of articular chondrocytes treated with 0.6 mM palmitate (P) and 0.4 mM oleate. DRB, siSTAMP2 and siFSP27 treatment significantly increased the FFA level in the cytosol compared with that in the experimental control (n=4). ** P<0.01 versus experimental control according to the Kruskal-Wallis nonparametric test. (D) The FFA level in the cytosol of articular chondrocytes treated with palmitate 1.6 mM palmitate and 0.4 mM oleate. Cilostazol significantly decreased the FFA level in the cytosol compared with that in the experimental control (n=4). ** P<0.01 versus experimental control according to the Kruskal-Wallis nonparametric test.

FIG. 9. Representative Nile red and BODIPY stainings. DRB pre-treatment reduces LD size and LD accumulation in cells treated with 1 mM oleate (O). Marked areas are magnified in FIG. 4D. Scale bar, 20 μm.

FIG. 10. Representative Nile red and BODIPY stainings. 30 μM Cilostazol (Cilo) increases LD size and LD accumulation in cells treated with 1.5 mM oleate (O). Marked areas are magnified in FIG. 4H. Scale bar, 20 μm.

FIG. 11. Representative with Nile red and BODIPY stainings. Knock-down of STAMP2 (A) or FSP27 (B) markedly reduces LD size and LD accumulation in cells treated with 1 mM oleate (O). Marked areas are magnified in FIG. 5D. Scale bars, 20 μm.

FIGS. 12A to 12D. TNF-α protected oleate-caused lipotoxicity through STAMP2. (A) Representative western blots showing that 25 ng/ml TNF-α reversed 1.5 mM oleate (0)-induced downregulation of STAMP2 protein (n=4). (B) Viability assay showing that 25 ng/ml TNF-α prevented 1.5 mM oleate-induced cell death (n=4). ** P<0.01 versus 1.5 mM oleate alone treatment; Kruskal-Wallis nonparametric test. (C) Representative phase contrast microscopic findings showing that 25 ng/ml TNF-α prevented oleate-induced cell death (n=4). Scale bar, 20 μm. (D) TNF-α reversed 1.5 mM oleate-induced reduction of LD size and total LD volume. ** P<0.01 versus 1.5 mM oleate alone treatment; Kruskal-Wallis nonparametric test. Scale bar, 10 μm.

### DETAILED DESCRIPTION

Exemplary embodiments of the present invention will be described in detail below with reference to the accompanying drawings. While the present invention is shown and described in connection with exemplary embodiments thereof, it will be apparent to those skilled in the art that various modifications can be made without departing from the scope of the invention.

Unless specifically stated otherwise, all the technical and scientific terms used in this specification have the same meanings as what are generally understood by a person skilled in the related art to which the present invention belongs. In general, the nomenclatures used in this specification and the experimental methods described below are widely known and generally used in the related art.

The present invention provides a method for treating or preventing osteoarthritis in a subject, comprising promoting the expression of STAMP2 (Six-transmembrane protein of prostate 2) in chondrocyte.

By promoting the expression of STAMP2 in the chondrocyte, the method of the present invention may be used to protect articular chondrocytes against FFAs-induced lipoapotosis, thereby preventing or treating osteoarthritis. And PKCK2/STAMP2/FSP27-mediated LD accumulation in the articular chondrocytes protects chondrocyte against lipotoxicity.

In the present invention, PKCK2 participates in a series of complex cellular functions, including cell growth and proliferation, by catalyzing the phosphorylation of a large number of proteins. CK2 involves in FFAs-induced lipotoxicity or LD accumulation and that cilostazol prevents FFAs-induced lipotoxicity.

In the present invention, STAMP2 plays a pivotal role in lipid homeostasis and dysregulation of STAMP2 was implicated in metabolic and inflammatory diseases. STAMP2 protect articular chondrocytes against FFAs-induced lipoapotosis in metabolic OA.

In the present invention, FSP27, also known as CIDEC, belongs to cell death-inducing DNA fragmentation factor 45 (DFF45)-like effector (CIDE) family of proteins. FSP27 is involved in protecting articular chondrocytes against FFAs-induced lipoapotosis in metabolic OA through LD accumulation.

In some embodiments of the present invention, the method of the present invention includes promoting the expression of STAMP2 by administering cilostazol, TNF-α, or a vehicle into which a gene encoding STAMP2 is introduced, to a subject.

In the present invention, the vehicle is an adeno-associated virus, a retrovirus, a lentivirus, a herpes simplex virus, an alpha virus, etc., and preferably an adenovirus.

In the present invention, “administering” may be used without limitation as long as the composition according to one exemplary embodiment of the present invention can reach target tissues. For example, the administration method encompasses oral administration, intraarterial injection, intravenous injection, percutaneous injection, intranasal administration, transbronchial administration, or intramuscular administration.

Also, the present invention provides a method for treating a subject having osteoarthritis by administering an effective amount of a vehicle, which a gene encoding STAMP2 is introduced.

In yet another embodiment of the present invention, the pharmaceutical composition of the present invention may include the vehicle into which a gene encoding STAMP2 is introduced.

The term “subject” refers to an animal, preferably a mammal, and most preferably a human, who is the object of treatment, observation or experiment. The mammal may be selected from the group consisting of mice, rats, hamsters, gerbils, rabbits, guinea pigs, dogs, cats, sheep, goats, cows, horses, giraffes, platypuses, primates, such as monkeys, chimpanzees, and apes. In some embodiments, the subject is a human.

The term “effective amount” of a compound refers to a sufficient amount of the compound that provides a desired effect but with no- or acceptable-toxicity. This amount may vary from subject to subject, depending on the species, age, and physical condition of the subject, the severity of the disease that is being treated, the particular compound used, its mode of administration, and the like. A suitable effective amount may be determined by one of ordinary skill in the art.

The gene encoding STAMP2 is a gene encoding STAMP2 derived from the subject. In this case, genes known in the related art may be used as the gene encoding STAMP2.

Vehicles known in the related art may be used without limitation as the vehicle delivering the gene encoding STAMP2, and may, for example, include a liposome, a plasmid vector, a cosmid vector, a bacteriophage vector, a viral vector, etc., and preferably a viral vector.

Specific examples of the viral vector may include an adenovirus, an adeno-associated virus, a retrovirus, a lentivirus, a herpes simplex virus, an alpha virus, etc., and preferably an adenovirus.

In some embodiments, the vehicle into which the gene encoding STAMP2 is introduced described herein is administered systemically. As used herein, “systemic administration” refers to any means by which the compounds described herein can be made systemically available. In some embodiments, systemic administration encompasses intravenous administration, intraperitoneal administration, intramuscular administration, intracoronary administration, intraarterial administration (e.g., into a carotid artery), intradermal administration, subcutaneous administration, transdermal delivery, intratracheal administration, subcutaneous administration, intraarticular administration, intraventricular administration, inhalation (e. g., aerosol), intracerebral, nasal, naval, oral, intraocular, pulmonary administration, impregnation of a catheter, by suppository and direct injection into a tissue, or systemically absorbed topical or mucosal administration. Mucosal administration includes administration to the respiratory tissue, e.g., by inhalation, nasal drops, ocular drop, etc.; anal or vaginal routes of administration, e.g., by suppositories; and the like. In some embodiments, the compounds described herein are administered intravenously.

The dosage may be adjusted according to factors such as a formulation method, a mode of administration, the age, weight and sex of a subject, a degree of severity of a disease, a diet, an administration time, a route of administration, a secretion rate, and the susceptibility to response. According to one embodiment in which the viral vector is used, the viral vector may be intravenously administered at a dose of 1×10⁸ to 1×10¹¹ plaque-forming units (pfus).

Pharmaceutical formulations suitable for use with the present invention may also include excipients, preservatives, pharmaceutically acceptable carriers and combinations thereof the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Also, the present invention provides a method for diagnosing osteoarthritis, comprising following steps: a) measuring an expression level of STAMP2 or an amount of STAMP2 protein in chondrocyte from the subject; and b) comparing the measured results in step a) with that in a control sample.

In the present invention, the expression level of STAMP2 is measured using any one selected from the group consisting of RT-PCR, Competitive RT-PCR, Realtime RT-PCR, RPA (RNaseprotection assay), Northern blotting and DNA chip.

In the present invention, the amount of STAMP2 protein is measured using any one selected from the group consisting of Western blot, ELISA (enzyme linked immunosorbent assay), RIA (radioimmunoassay), radioimmunodiffusion, Ouchterlony immunodiffusion, rocket immunoelectrophoresis, IHC (immunohistochemistry), Immunoprecipitation assay, Complement fixation assay, FACS (fluorescence activated cell sorter) and protein chip.

Also, the present invention provides a method for screening a therapeutic agent for osteoarthritis, comprising following steps: a) treating chondrocyte with a candidate agent; b) measuring an expression level of STAMP2 or an amount of STAMP2 protein in the chondrocyte treated with the candidate agent; and c) identifying a candidate agent as the therapeutic agent when the expression level of STAMP2 or the amount of STAMP2 protein measured in step b) is increased in the chondrocyte as compared to the chondrocyte before treatment with the candidate agent.

Hereinafter, preferred Examples are provided to aid in understanding the present invention. However, it should be understood that detailed description provided herein is merely intended to provide a better understanding of the present invention and is not intended to limit the scope of the present invention.

### EXAMPLES

1: Preparation and Method

1-1. Animals

All procedures for animal study were approved by the Committee on Animal Investigations at Dong-A University (DIACUC-15-12). Seven-week-old male C57BL/6 mice were obtained from Samtoko (Osan, Korea). Mice were maintained in a temperature-controlled room (22° C.) on a 12:12 h light-dark cycle and free access to water and food.

1-2. Reagents

All The following reagents were obtained commercially: goat polyclonal anti-human CKIIα and HRP-conjugated donkey anti-goat IgGs antibodies from Santa Cruz Biotechnology (Santa Cruz, Calif., USA); rabbit polyclonal anti-human caspase-3 and -7 antibodies from Cell Signaling (Danvers, Mass., USA); rabbit polyclonal anti-human STAMP2 antibody from Proteintech (Rosemont, Ill., USA); mouse monoclonal anti-FSP27 antibody from Abcam (Cambridge, Mass., USA); HRP-conjugated donkey anti-rabbit and sheep anti-mouse IgGs from Amersham Pharmacia Biotech (Piscataway, N.J., USA); Ketamine hydrochloride from Sanofi-Ceva (Dusseldorf, Germany); Rompun from Bayer (Leverkusen, Germany); Dulbecco's modified Eagle's medium (DMEM) and fetal bovine serum (FBS) from Gibco BRL (Gaithersburg, Md., USA); TNF-α and ApopTag FITC In Situ Apoptosis Detection Kit from Millipore (Temecular, CA); mouse monoclonal anti-human actin antibody, Hoechst 33342, dimethylsulfoxide (DMSO), RNase A, proteinase K, aprotinin, leupeptin, propidium iodide (PI), phenylmethylsulfonyl fluoride (PMSF), protein-A agarose and 5,6-dichlorobenzimidazol riboside (DRB), fatty acid-free bovine serum albumin (BSA), Oil red 0, palmitate, oleate, stearic acid (SA), mono-iodoacetate (MIA), 3,3′-diaminobenzidine (DAB), cilostazol and type II collagenase from Sigma (St. Louis, Mo., USA); caspase inhibitor I (zVAD-fmk) from Calbiochem (San Diego, Calif., USA); 5,5′,6,6′-tetrachloro-1,1′,3,3′-tetraethylbenzimidazol carbocyanine iodide (JC-1), Nile red and BODIPY 493/503 from Molecular Probes (Eugene, Oreg., USA); SuperSignal WestPico enhanced chemiluminescence western blotting detection reagent from Pierce (Rockford, Ill., USA).

1-3. Osteoarthritis (OA) Models

Mice subjected to surgery for experimental OA. Sixty mice were used. After a normal chow diet for one week, half of mice were fed HFD and the other half SD. Mouse fed an HFD or a SD for 8 weeks was subjected to surgery for experimental OA. Mice were anesthetized with ketamine hydrochloride (15 mg/kg) and Rompun (3.45 mg/kg) and knees were prepared for aseptic surgery. The skin incision for cutting of anterior cruciate ligament was a 5 mm longitudinal incision over the distal patella to proximal tibial plateau. The joint capsule, which site was located medial to the patellar tendon, was incised with a #15 blade and the joint capsule opened with micro-iris scissors. Blunt dissection of the fat pad over the intercondylar area was performed to expose either the intercondylar region, providing visualization of the anterior cruciate ligament. The anterior cruciate ligament was transected with a micro-surgical knife under direct visualization and complete transection confirmed by the presence of anterior drawer. After anesthesia was released, mice had excellent mobility within 2 h after either surgery. At 4, 6 and 8 weeks after surgery, ten mice from each diet were used for histological evaluation.

HFD-induced OA mouse model. Sixty mice were fed a normal chow diet for one week and then mice were fed an HFD or a SD for 25 weeks. Ten mice from each diet were used histologically examine the effect of high fat diet. Forty mice were used to examine the effect of cilostazol. Mice fed a SD or HFD for 15 weeks were orally administered cilostazol at concentrations of 30 mg/kg/day for additional 10 weeks. SD+vehicle mice (n=10) were fed a SD and received DMSO. SD+cilostazol mice (n=10) were fed a SD and received cilostazol. HFD+vehicle mice (n=10) were fed a HFD and received DMSO. HFD+cilostazol mice (n=10) were fed a HFD and received cilostazol.

1-4. Tissue Preparation and Histologic Examination

Tissue preparation and histologic examination. Animals were killed by ether inhalation. Whole knee joints were removed by dissection, fixed in in PBS (pH 7.4) containing 4% paraformaldehyde, decalcified in 12.5% EDTA, dehydrated, and embedded in paraffin blocks. Five-micrometer microsections were prepared and stained with hematoxylin and eosin and with Safranin O-fast green.

Immunohistochemical observation and analysis. Tissue sections were incubated in 1:70-diluted goat serum solution for 30 minutes at room temperature, and then for 2 hours with 1:100-diluted primary antibody at room temperature. Next, sections were incubated with secondary antibody for 1 hour at 37° C. and developed using ABC complex. Peroxidase was revealed by DAB and examined by light microscopy. Histological images were observed and analyzed using a Aperio ScanScope® CS system. Total numbers of positive for PKCK2, FSP27 and STAMP2 in four fields per animal were counted by an observer blinded to the experiment and the percentage of positive cells were calculated.

1-5. Cell Culture of Articular Chondrocytes

Rat articular chondrocytes for primary culture were isolated from knee joint cartilage slices by enzymatic digestion for 1 h with 0.2% type II collagenase (381 units/mg) in DMEM. After the isolated cells were collected by brief centrifugation, they were resuspended in DMEM supplemented with 10% (v/v) FBS, 50 mg/ml streptomycin and 50 units/ml penicillin (Gibco). The cells were plated on culture dishes at a density of 5×10⁴ cells/cm². The medium was replaced every 2 days, and they reached confluence after approximately 5 days in culture. In each experiment, the cells from three animals were pooled and analyzed three times.

1-6. Treatment of Free Fatty Acids or Combination Treatment with Other Chemicals.

FFA were dissolved in absolute ethanol at a concentration of 500 mM and diluted to final concentrations of FFAs with the appropriate concentration of 1% (w/v) FFA-free BSA. Controls were incubated with equal concentrations of FFA-free BSA containing ethanol. To examine the effect of several chemicals, cells were pretreated with 150 μg/mL DRB or 30 μM cilostazol for 24 h or 25 ng/ml TNF-α for 3 h before FFA treatment.

1-7. siRNA

Rat STAMP2 and FSP27 siRNA. Rat STAMP2 siRNA (SMART pool; L-105419-02-0050) and FSP27 siRNA (SMART pool; L-105647-02-0050) were purchased from Thermo Scientific (Hudson, N.H., USA). As a negative control, the same nucleotides were scrambled to form nongenomic combinations.

siRNA transfection or combination treatment with FFAs. Transfection of siRNA was performed with the use of siPORT Amine and Opti-MEM media. Cells grown to a confluence of 40% to 50% in six-well plates were transfected with 100 nM final siRNA concentration per well. Transfection mixture was added to each well, and incubation occurred for 4 h. Then 2 ml of growth medium was added, and cells were incubated for another 20 h. After siRNA transfection medium was removed, each well was washed in PBS solution. Cells were treated with FFAs for 24 hours.

1-8. Recombinant Adenoviral STAMP2 Infection

Recombinant adenoviral STAMP2 was prepared as described previously34. Cells (1×10⁷) were infected with recombinant adenoviral STAMP2 at multiplicities of infection (MOI) of 500, 1000 and 1500 to the medium.

1-9. Staining of Lipid Droplets, Confocal Microscopy and Quantification

Cells cultured on a coverslip were incubated with diluted Nile red or BODIPY 493/503. Some sections were double-labelled with TUNEL and BODIPY 493/503 and counterstained with Hoechst 33342. Fluorescence images were observed and analyzed using a Zeiss LSM 510 laser-scanning confocal microscope (Goettingen, Germany). Twenty cells from each experiment were observed and auantification of fluorescence intensity of the confocal images were obtained with the use of ImageJ software. Fluorescence intensity was expressed as AU.

1-10. Oil Red O Staining

Cells were washed twice in PBS and fixed for 1 h with 10% (w/v) formaldehyde in PBS. After two washes in 60% isopropyl alcohol, the cells were stained for 30 min in freshly diluted Oil Red O solution. Then, the stain was removed, and the cells were washed four times in water.

1-11. Total Cytosol FFA Content Measurement

Cell were collected after treatment with trypsin (0.2% trypsin, 0.02% EDTA and 0.2% glucose in PBS) and pelleted by centrifugation (200×g for 5 min at 4° C.). Cell pellet was resuspended in ice-cold hypotonic lysis medium containing 20 mM Tris-HCl, pH 7.4 and 1 mM EDTA. Cells were homogenized with a Dounce homogenizer and then centrifuged (800×g for 5 min at 4° C.). The post-nuclear supernatant fraction was ultra-centrifuged (800×g for 5 min at 4° C.) using Beckman Table-top ultracentrifuge. LD fraction with the distinct white band on the preparation was removed with a pipetman and LD-free cytosol was used for FFA measurement. Cytosol FFA levels were measured using a commercial free fatty acid quantification kit from Abcam (ab65341).

1-12. Cell Viability Assay

An automated trypan blue exclusion assay was undertaken. Total cells and trypan bluestained (i.e., nonviable) cells were counted, and the percentage of nonviable cells was calculated using the Vi-Cell cell counter (Beckman Coulter, Miami, Fla., USA).

1-13. Nuclear Morphology Study for Apoptosis

Cells were harvested and then washed with PBS. They were fixed in 4% paraformaldehyde for 20 min at room temperature. The cells were washed with PBS twice, and stained in 4 μg/ml Hoechst 33342 for 1 h at 37° C. Stained cells were coated onto clean, lipid-free glass slides and mounted with a cover glass. The samples were observed and photographed under an epifluorescence microscope (Axiophot, Zeiss, Germany).

1-14. Quantification of DNA Hypoploidy and Cell Cycle Phase Analysis by Flow Cytometry

Cells were washed twice with PBS, and fixed with cold 70% ethanol at 4° C. overnight. The fixed cells were pelleted and ethanol was removed by washing twice with PBS containing 1% bovine serum albumin (BSA). The cells were resuspended in 1 ml of PBS containing 11 Kunitz U/ml RNase A, incubated at 4° C. for 30 min and washed once with BSAPBS. Cells were resuspended in PI solution (50 μg/ml) and incubated at 37° C. for 30 min in dark. Cells were washed with PBS, the DNA content of 10,000 cells was used for the generation of simultaneous estimation of the cell cycle parameters and apoptosis using an Epics XL (Beckman Coulter, FL).

1-15. Western Blot Analysis

Cell (2×10⁶) were washed twice with ice-cold PBS. Cells were resuspended in lysis buffer [200 ll of icecold solubilizing buffer (300 mM NaCl, 50 mM Tris-Cl (pH 7.6), 0.5% Triton X-100, protease inhibitor cocktail)] and incubated at 4° C. for 30 min. The lysates were centrifuged at 14,000 rpm for 20 min at 4° C. The protein concentrations of the cell lysates were measured with the Bradford protein assay reagent (Bio-Rad). Then, 50 μg of proteins was loaded onto 15 SDS-PAGE. The separated proteins were transferred to nitrocellulose membranes (Amersham Pharmacia Biotech, Piscataway, N.J., USA) and probed with each antibody. Immunostaining with the antibodies was carried out using the Super Signal West Pico enhanced chemiluminescence substrate and detected with LAS-3000PLUS.

1-16. Assay of Mitochondrial Membrane Potential (MMP)

Disruption of mitochondrial membrane potential (MMP) was measured using a specific fluorescent probe, JC-1, that was added directly to the cell culture medium (5 μg/mL final concentration) and incubated for 15 minutes at 37° C. Cells were stained with JC-1, and flow cytometry to measure MMP was performed (an Epics XL; Beckman Coulter). Data were acquired and analyzed using EXP032 ADC XL 4 color software.

1-17. TUNEL Staining of Cell Suspensions

Cell suspensions were cytospun onto clean fat-free glass slides in a cytocentrifuge. After being fixed with 4% paraformaldehyde, the cells were incubated with terminal deoxynucleotidyl transferase (TdT) enzyme for 1 h at 37° C., and antidigoxigenin-FITC was applied for 30 min at room temperature. Afterward the cells were incubated with a PI primary antibody for 1 h at 37° C. Nuclei were counterstained with DRAQ5™. Fluorescent images were observed and analyzed using a Zeiss LSM 510 laser-scanning confocal microscope.

1-18. Statistics

Four independent experiments were carried out in vitro. The results are expressed as means S.D. from four experiments, each performed in triplicate. The results of the experimental and control groups were tested for statistical significance by the Kruskal-Wallis nonparametric test. To test a statistical significance for a difference in the occurrences of the onset of OA between HFD and SD, we first made two-by-three contingency table where HFD/SD is in the row and 4/6/8 weeks are in the column and Pearson's χ^{̂2} test was conducted to analyze an association between diet and time. In order to test the significance of an individual variable, we next fitted a Poisson regression model where the occurrences of the onset of OA among 10 mice is on the response and both two different types of diet (HFD/SD) and three different weeks (4/6/8) are on the predictors. The likelihood ratio test for a diet variable was conducted. For the comparison between HFD and SD for 25 weeks without surgery, the Chi-squared test for the same probabilities of two groups was conducted (P<0.01).

2: Results

2-1. HFD Accelerates the Onset of OA.

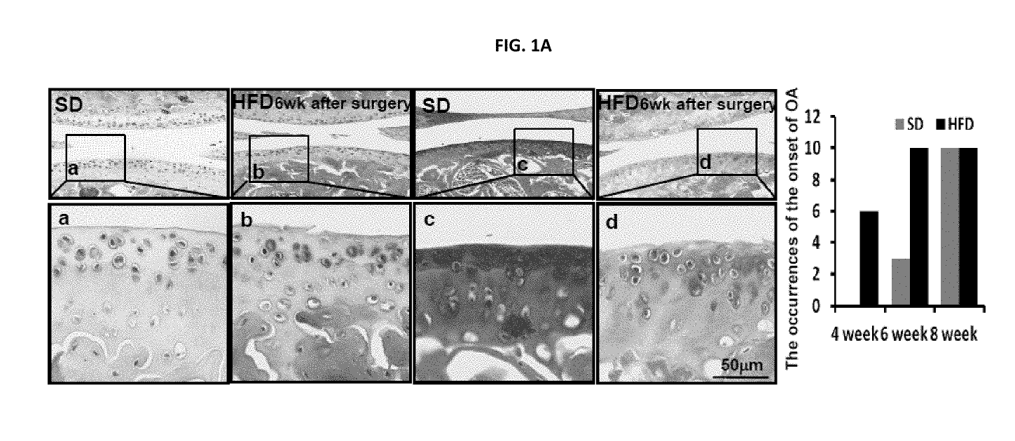

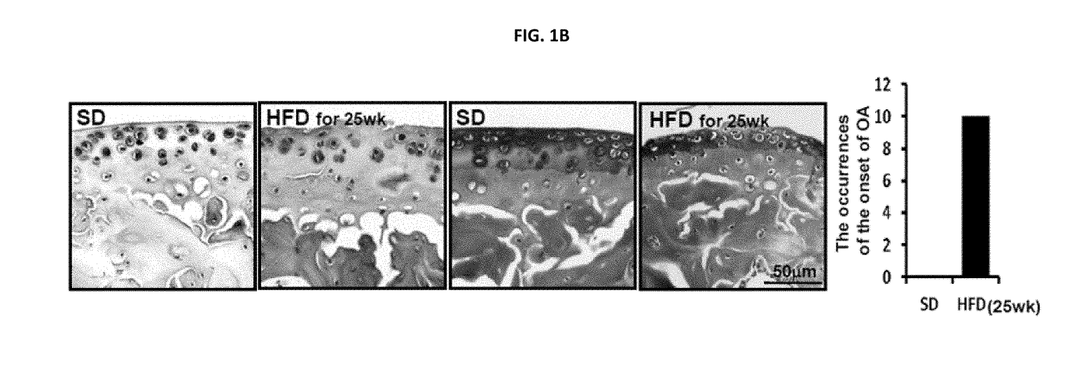

Using two experimental mouse OA models, we examined whether a high-fat diet (HFD) accelerated the onset of OA. The onset of OA was determined by histological findings characteristic of OA, such as an irregular surface, the disappearance of surface layer cells, and reduced Safranin O staining. First, mice fed a HFD or a standard diet (SD) for 12 weeks were subjected to surgery for experimental OA, and after the indicated time, cartilages were histologically observed. We could observe the onset of OA 4 weeks (6/10) or at least 6 weeks (10/10) after surgery in mice fed a HFD. In contrast, these findings were not observed in any mouse fed a SD 4 weeks after surgery, while these findings were observed at 6 weeks (3/10) and 8 weeks (10/10) after surgery in mice fed a SD (FIG. 1A). There was a statistically significant difference between the HFD and SD based on the likelihood ratio test of the Poisson regression model. Furthermore, we examined the onset of OA findings in mice fed a HFD or a SD for 25 weeks without surgery for experimental OA. We observed characteristic OA findings in all mice fed a HFD (10/10) but not in any mouse fed a SD (0/10) (FIG. 1B). Indeed, there was statistical significance for the probability of OA findings between mice fed a HFD and a SD based on the chi-squared test (P<0.01). These findings indicate that HFD feeding accelerates the onset of OA.

2-2. Palmitate but not Oleate at Usual Clinical Ranges Exerts Lipotoxicity, while Oleate at High Pathological Ranges Exerts Lipotoxicity in Rat Articular Chondrocytes Through Apoptosis.

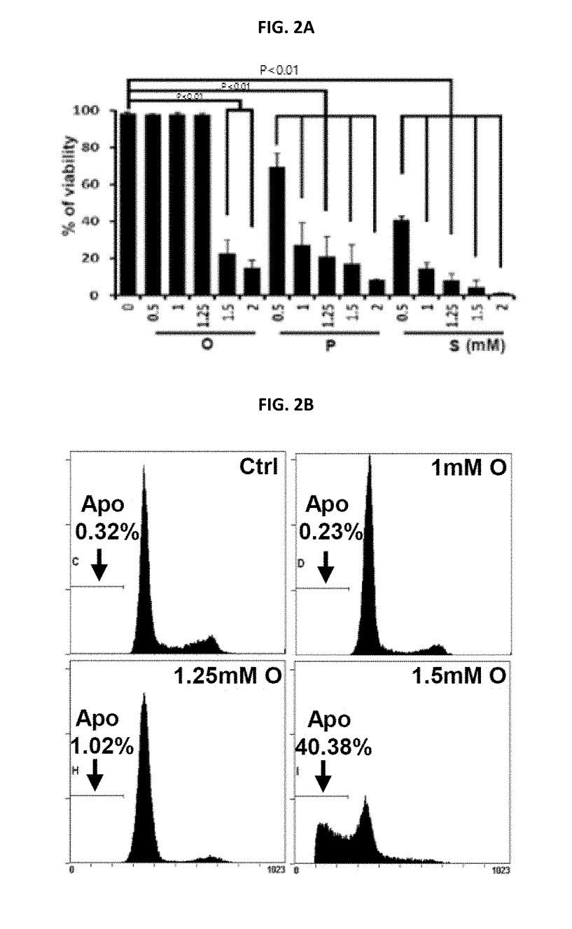

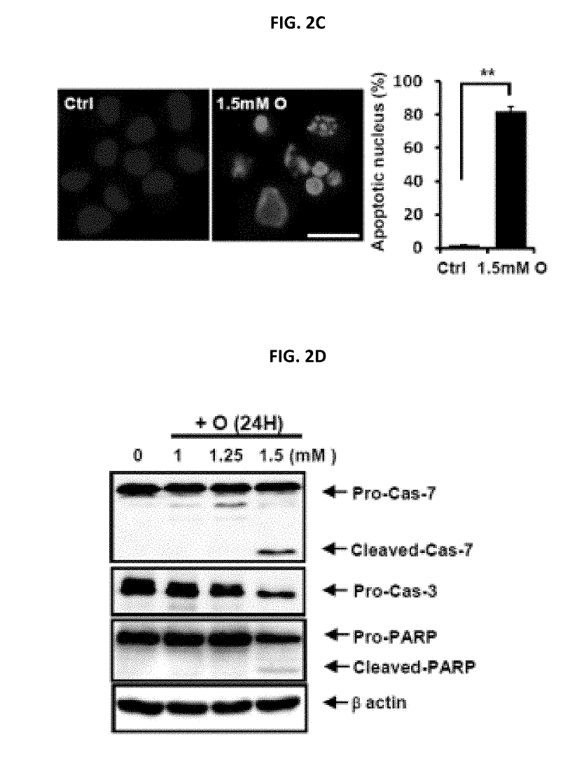

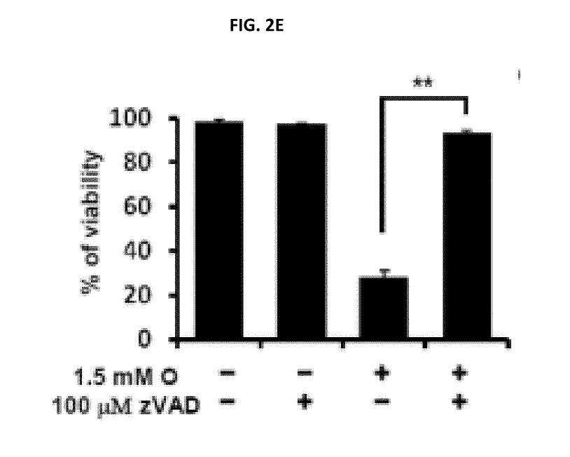

We next examined whether FFAs exert lipotoxicity in rat articular chondrocytes. Although several studies have suggested that there is an increased FFA concentration in OA synovial fluid, no documented studies have reported the FFA levels in the synovial fluid of OA subjects. Although FFAs have been extensively used at concentrations of 50 to 750 μM in experimental studies, the FFA levels in plasma are usually only 0.2 to 2 mM. Thus, in the present study, we screened the effect of FFAs at 0.1 to 2 mM on rat articular chondrocyte viability. We observed that palmitate or stearate at 0.5 to 2 mM reduced the viability of rat articular chondrocytes. While treatment at 0.5 to 1.25 mM did not reduce cell viability, oleate at 1.5 to 2.0 mM significantly reduced the viability of rat articular chondrocytes (FIG. 2A). Although most previous reports using oleate under 1.0 mM found that oleate did not exert lipotoxicity, our findings suggest that oleate at high pathological ranges exerts lipotoxicity in articular chondrocytes. As the mechanism underlying the lipotoxicity exerted by this toxic concentration of oleate might offer insight into the defence mechanism of nontoxic concentrations of oleate, we next examined the underlying mechanism by which toxic concentrations of oleate exert lipotoxicity. Various apoptosis assays demonstrated that toxic concentrations of oleate exerted lipotoxicity in articular chondrocytes through apoptosis (FIGS. 2B to 2E).

2-3. LD Accumulation Through FSP27 is Associated with the Resistance of Articular Chondrocytes to Oleate-Caused Lipotoxicity.

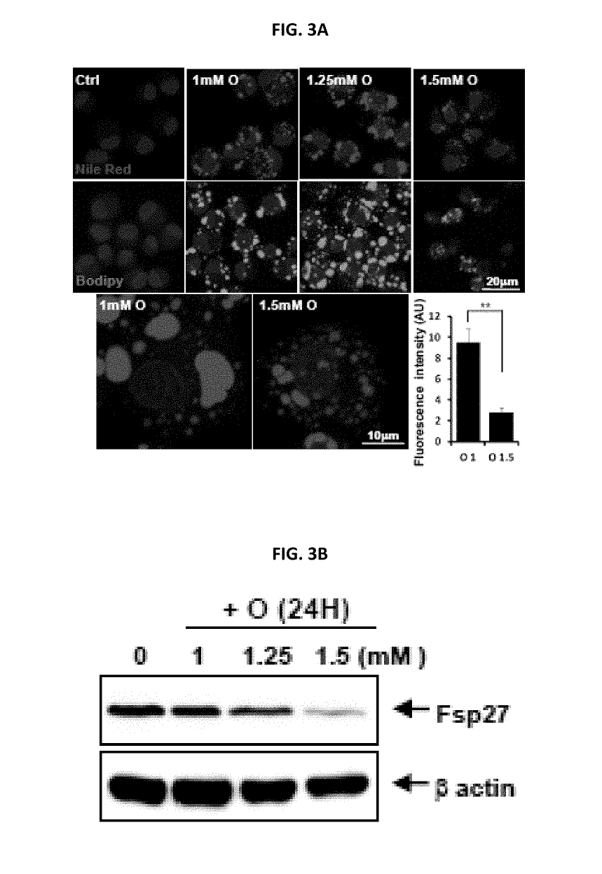

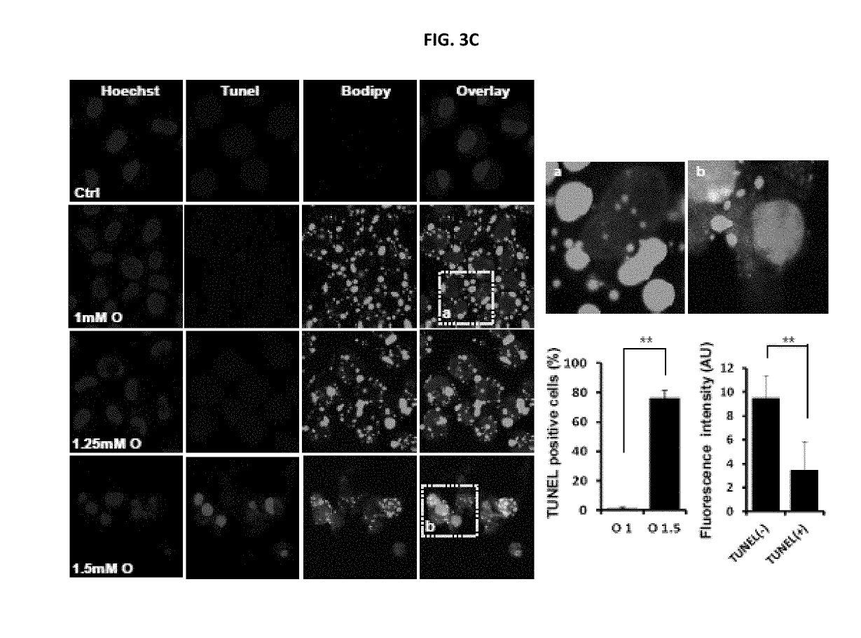

We analysed cells stained with BODIPY 493/503 using flow cytometry and observed an increase in size and granularity after 1 to 1.25 mM oleate treatment, which indicate the accumulation of neutral lipids. Noticeably, the size and granularity were decreased after 1.5 mM oleate treatment. Because these findings suggest that LD accumulation is associated with resistance to lipotoxicity, further studies focused on LD accumulation. Confocal microscopy demonstrated that giant LDs formed in chondrocytes treated with nontoxic concentrations of oleate. Importantly, toxic concentrations of oleate reduced the size of LDs and the total LD volume (FIG. 3A). Western blot assays showed that toxic concentrations of oleate decreased the expression level of fat-specific protein 27 (FSP27), which is associated with the surface of intracellular LDs (FIG. 3B). Giant LDs were not observed in TUNEL-positive cells. The total LD volume was significantly reduced in TUNEL-positive cells undergoing apoptosis compared with that of TUNEL-negative cells (FIG. 3C). These data indicate that LD accumulation is associated with the resistance of articular chondrocytes to oleate-induced lipotoxicity.

2-4. PKCK2 Inhibition Prohibits Oleate-Induced LD Accumulation, Resulting in Chondrocytes Death.

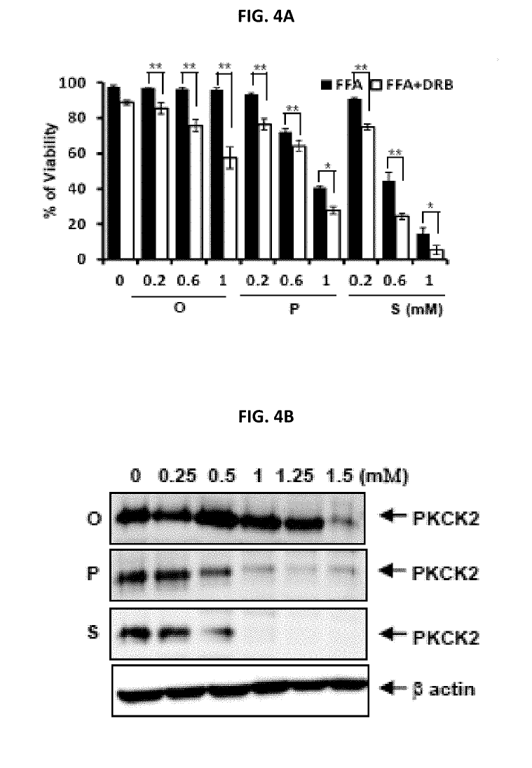

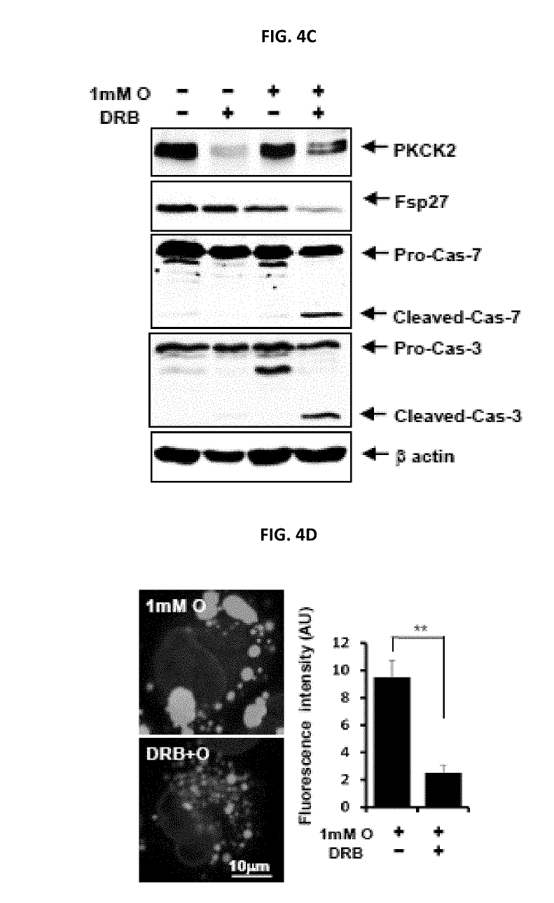

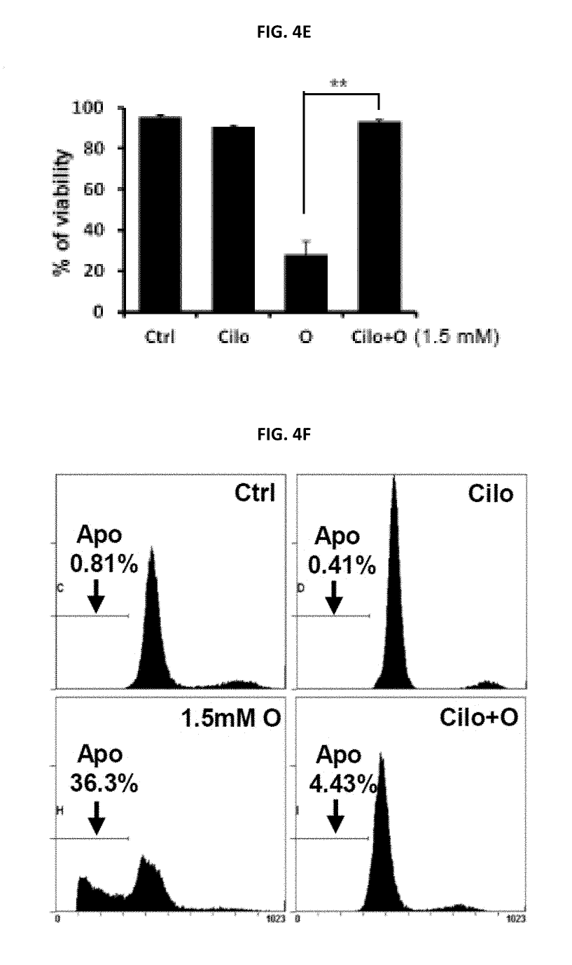

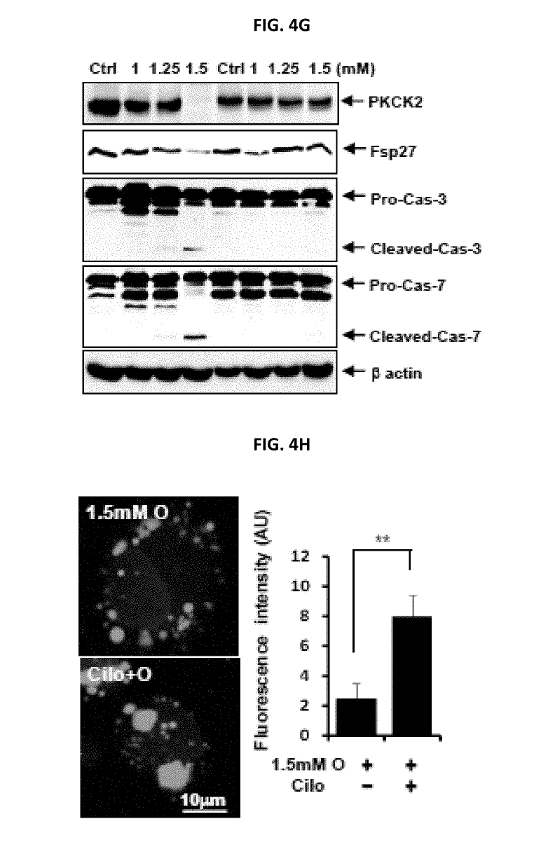

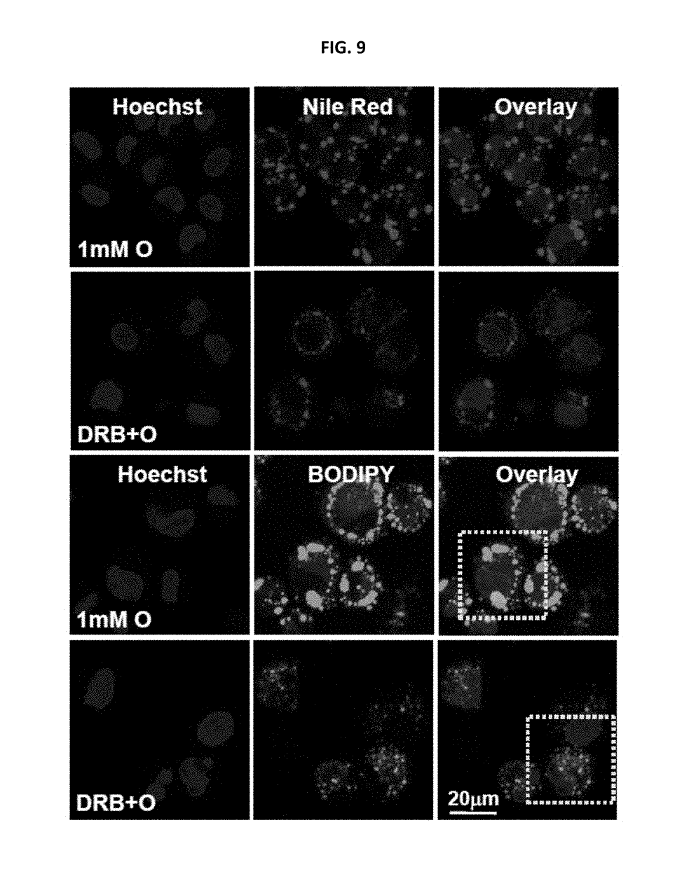

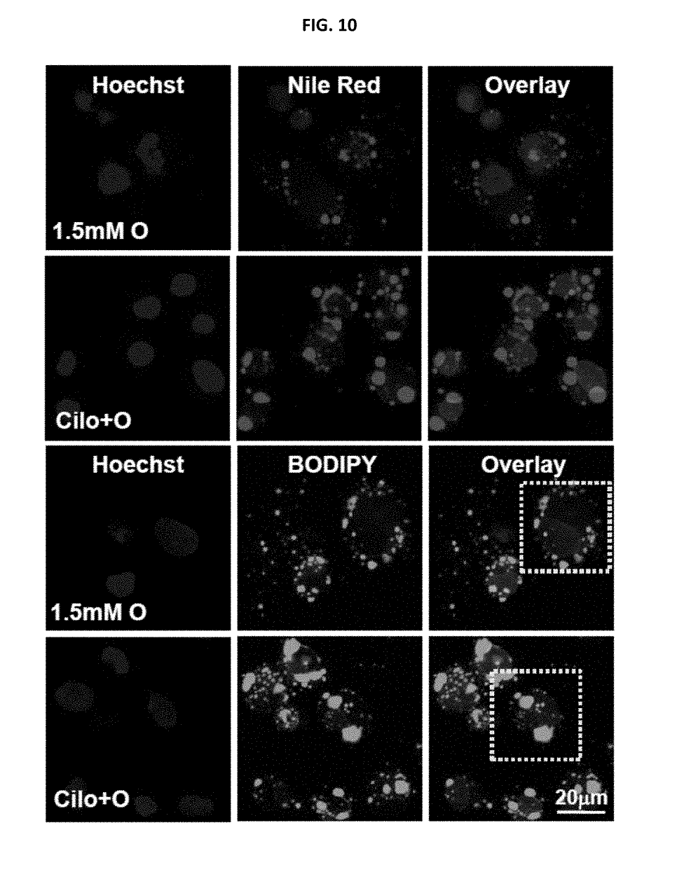

Elevated FFAs should act synergistically with destructive stimuli in the pathogenesis of OA. Because a previous study reported that FFAs augment chondrocyte death through IL-1-β, we further examined whether FFAs augment chondrocyte death by another representative stimulus known to induce articular chondrocytes, protein kinase casein kinase 2 (PKCK2). Our viability assay revealed that all types of FFA tested sensitized chondrocytes to cell death caused by 5,6-dichlorobenzimidazol riboside (DRB), a PKCK2 inhibitor (FIG. 4A). Because PKCK2 downregulation is an important signal inducing articular chondrocyte death, we further examined whether FFAs downregulate PKCK2. We observed that toxic concentrations of all FFAs tested reduced the expression level of PKCK2 protein. Noticeably, oleate at 1.5 mM, but not at 1 to 1.25 mM, markedly reduced the expression level of the PKCK2 protein (FIG. 4B). Western blot assays showed that 1 mM oleate with 100 μM DRB decreased the FSP27 protein level (FIG. 4C). We next examined whether 1 mM oleate with DRB augmented PKCK2 inhibition-induced cell death through apoptosis. Western blot assays demonstrated that 1 mM oleate in conjunction with DRB induced the activation of pro-apoptotic caspase-3 and -7 in articular chondrocytes (FIG. 4C). We also observed that co-treatment with DRB reversed 1 mM oleate-induced LD accumulation. Confocal microscopy and its quantification showed that the total LD volume was reduced in articular chondrocytes undergoing apoptosis induced by oleate plus DRB (FIG. 9 and FIG. 4D). These findings suggest that PKCK2 inhibition prohibits oleate-caused LD accumulation, resulting in the induction of cell death. Because cilostazol prevents the reduction of PKCK2 activity in rat articular chondrocytes, we next examined whether upregulation of PKCK2 by cilostazol protects articular chondrocytes to oleate-caused lipotoxicity. We observed that cilostazol pretreatment prevented oleate-caused lipoapoptosis (FIGS. 4E to 4G). Notably, cilostazol reversed oleate-caused decrease of FSP27 protein expression level (FIG. 4G). Confocal microscopy and its quantification showed that the prevention of chondrocytes death by cilostazol is associated with LD accumulation (FIG. 10 and FIG. 4H). These findings indicate that PKCK2 inhibition involves in reducing the total LD volume, triggering cell death.

2-5. STAMP2 Confers Articular Chondrocytes the Resistance to Oleate-Caused Lipotoxicity Through Maintaining LD Accumulation.

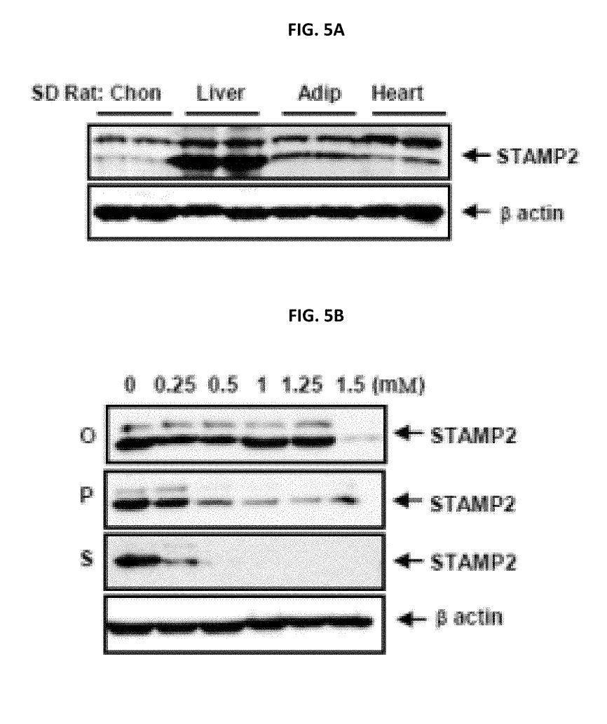

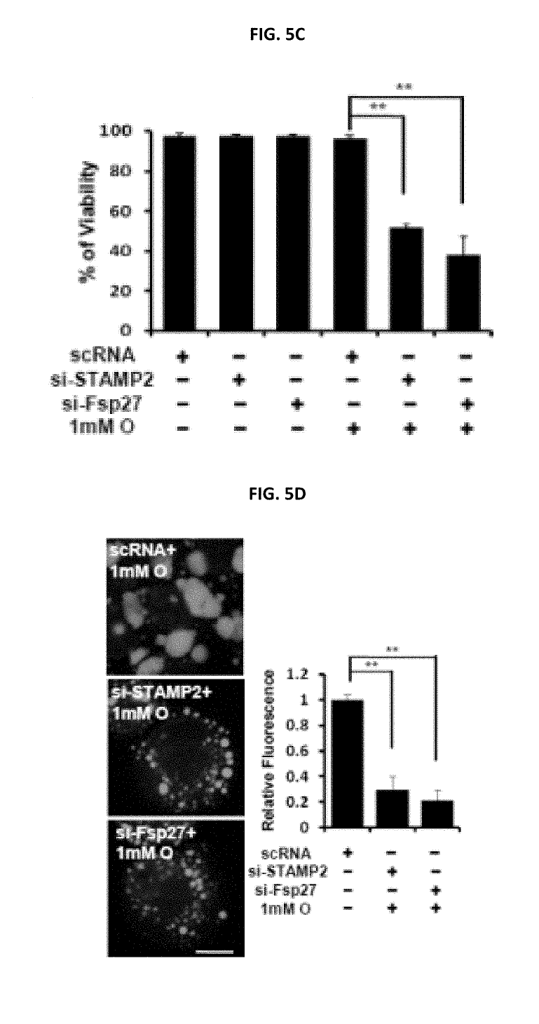

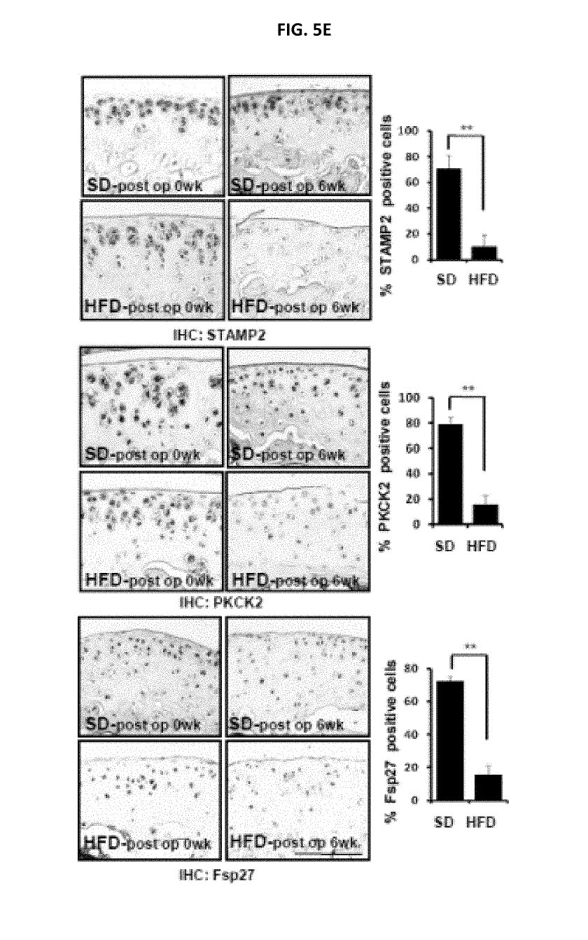

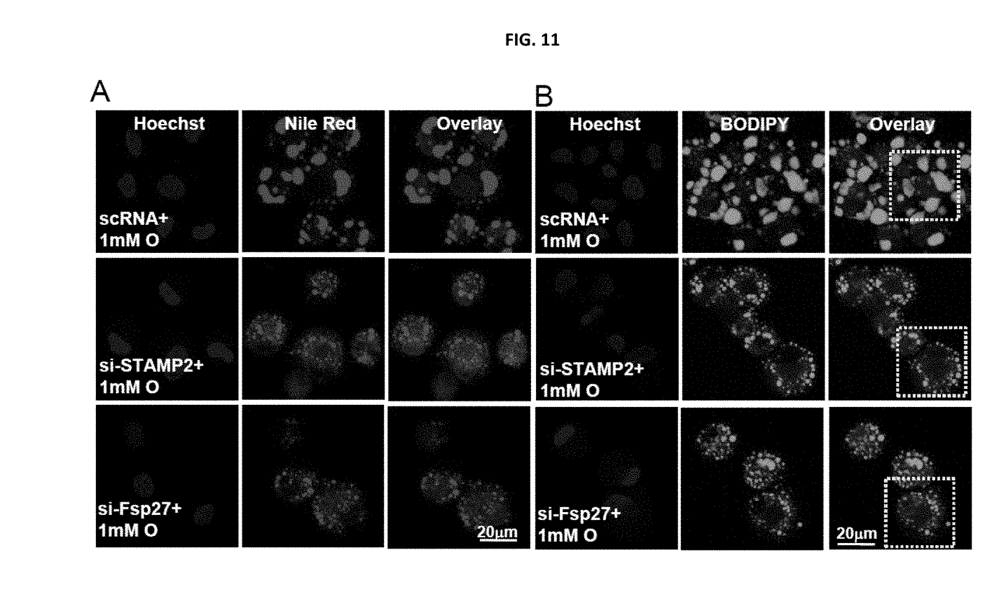

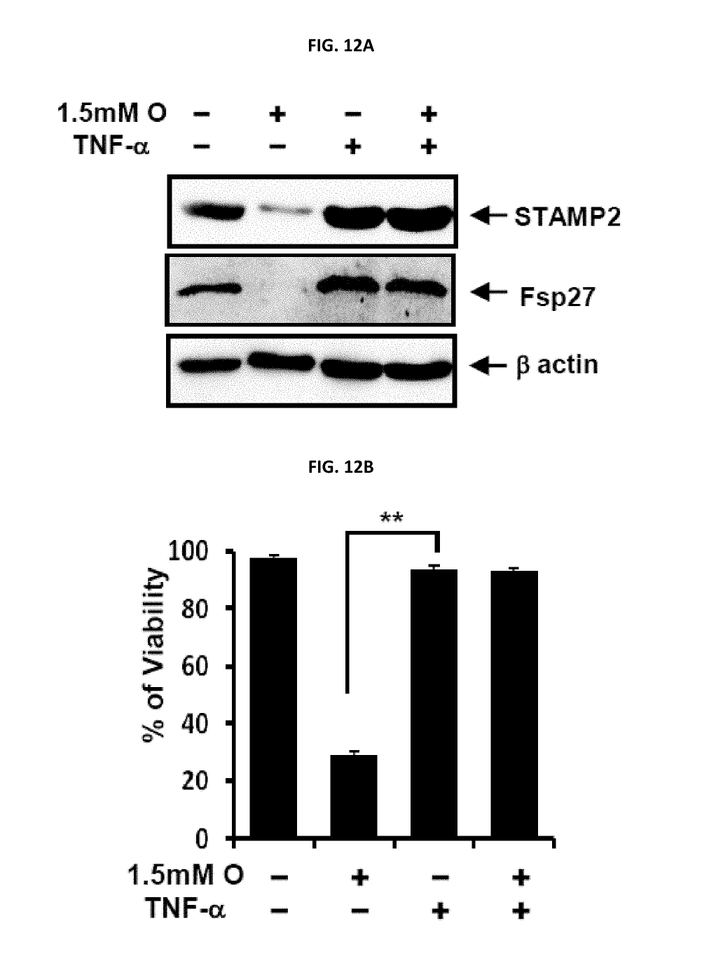

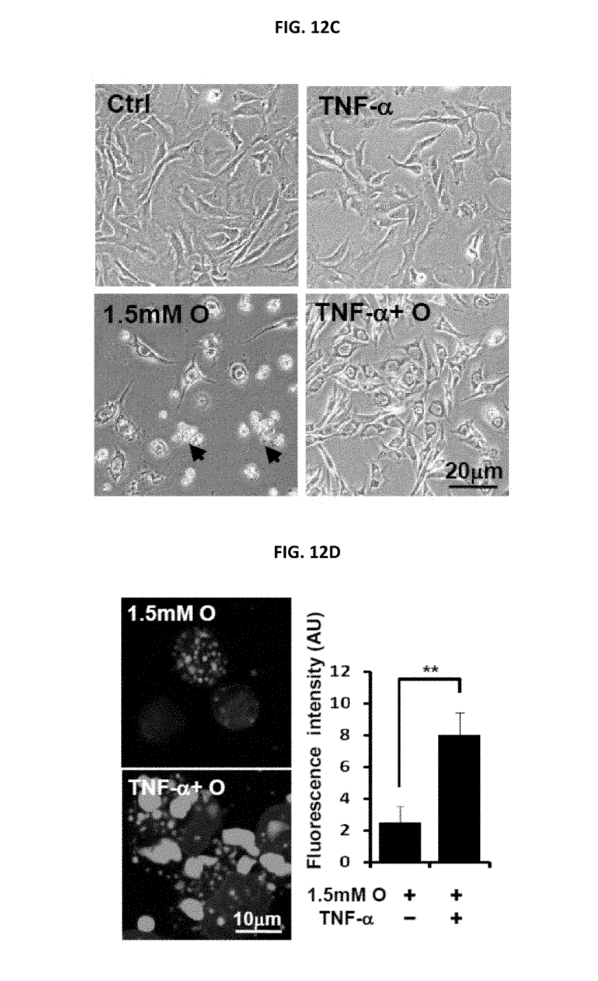

We observed for the first time that six transmembrane protein of prostate 2 (STAMP2), which plays a pivotal role in lipid homeostasis, is substantially expressed in rat articular chondrocytes in vitro and in vivo (FIG. 5A). Thus, we next examined whether STAMP2 involves in the pathophysiology of HFD-associated OA. We observed that toxic concentration of all FFAs tested markedly reduced the expression level of STAMP2 in vitro (FIG. 5B). We also examined the effect of knockdown of STAMP2 on oleate-induced lipotoxicity. Notably, 1 mM oleate substantially induced cell death in STAMP2-depleted chondrocytes, indicating that STAMP2 depletion sensitizes chondrocytes to lipotoxicity (FIG. 5C). In addition, we observed that the knockdown of STAMP2 markedly reduced the total LD volume (FIG. 11 and FIG. 5D). The reductions in the viability and total LD volume by the knockdown of STAMP2 were equivalent to those caused by knockdown of FSP27 (FIG. 5C and FIG. 5D). These data suggest that STAMP2 is involved in the resistance of chondrocytes to lipotoxicity through maintaining LD accumulation. We next examined the effect of TNF-α, which has been reported to upregulate STAMP2, on oleate-caused lipotoxicity. TNF-α completely reversed the 1.5 mM oleate-induced downregulation of the expression level of STAMP2 protein (FIG. 12A). A viability assay and phase contrast microscopy further showed that TNF-α completely reversed 1.5 mM oleate-induced cell death (FIG. 12B and FIG.-C). Furthermore, TNF-α completely reversed the 1.5 mM oleate-induced reduction in total LD volume (FIG. 12D). These findings suggest that STAMP2 confers articular chondrocytes resistance to lipotoxicity through maintaining LD accumulation. We further examined whether a HFD decreased the population of STAMP2-positive cells in vivo. Immunohistochemistry analysis of the experimental OA mouse model subjected to surgery demonstrated that the population of STAMP2-positive cells was significantly larger in mice fed a SD than in mice fed a HFD (FIG. 5E). We also observed that the population of PKCK2- or FSP27-positive articular chondrocytes was significantly larger in mice fed a SD than in mice fed a HFD (FIG. 5E).

2-6. PKCK2/STAMP2/FSP27 Axis Confers Articular Chondrocytes the Resistance to Lipotoxicity.

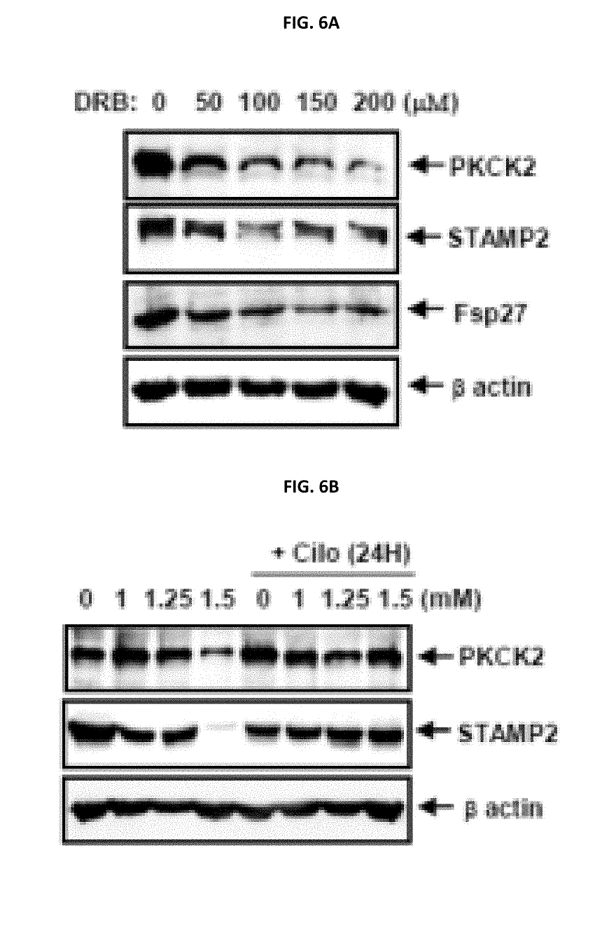

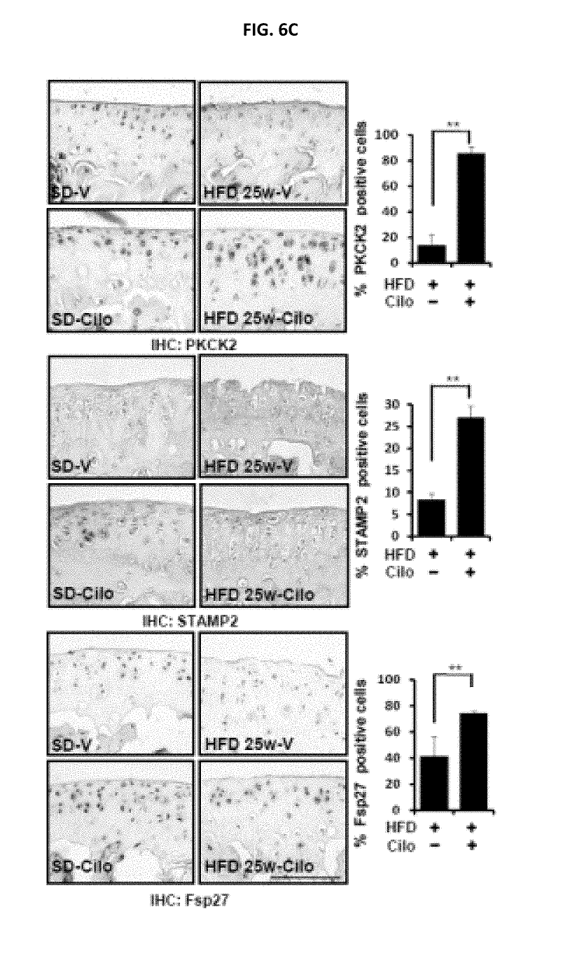

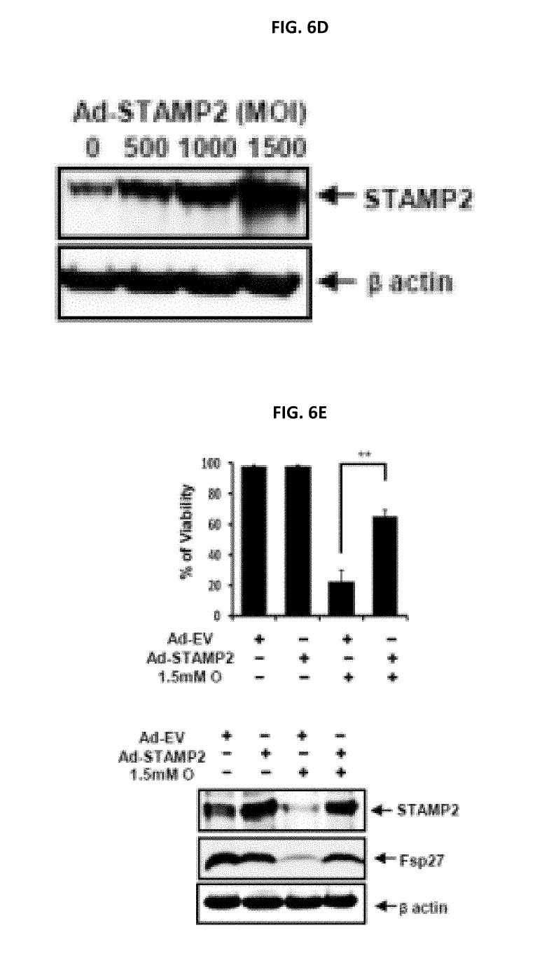

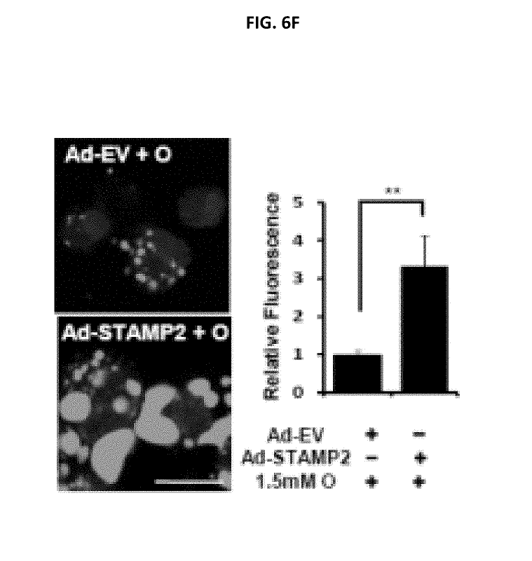

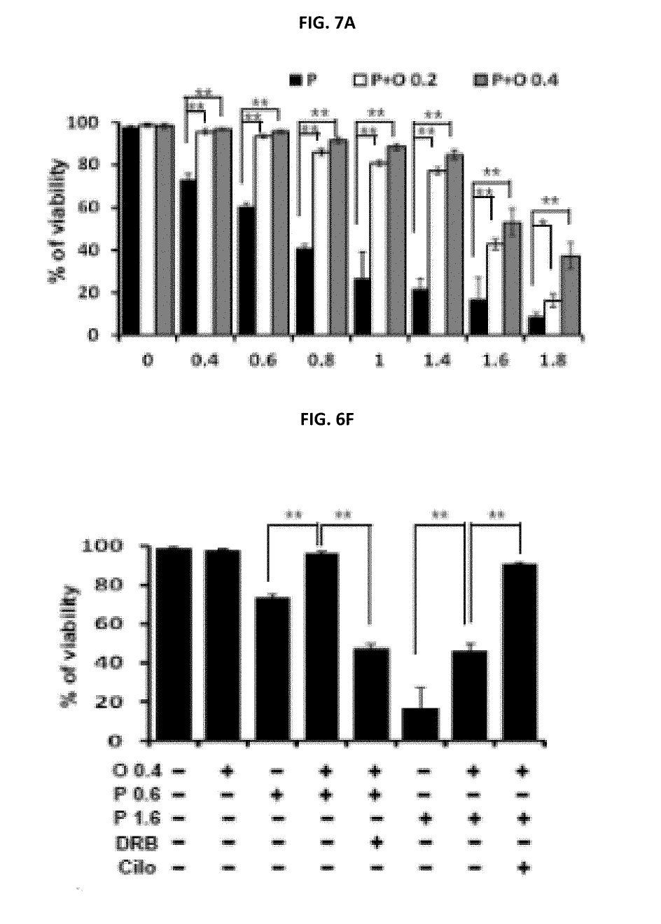

We next examined the hierarchical regulation of the resistance to lipotoxicity. We first observed that PKCK2 inhibition decreased the expression levels of FSP27 and STAMP2 in vitro (FIG. 6A). Furthermore, the PKCK2 augmenter cilostazol completely reversed the downregulation of STAMP2 induced by oleate in vitro (FIG. 6B). In conjunction with the data shown in FIG. 4G, these findings suggest that PKCK2 functions upstream of STAMP2 and FSP27. Thus, we next examined the effect of cilostazol on HFD-induced cartilage destruction using the OA model without surgery. We observed that cilostazol administration markedly prevented the HFD-induced cartilage destruction (FIG. 6C). We further examined the effect of cilostazol on the populations of PKCK2-, STAMP2- or FSP27-positive cells. Noticeably, cilostazol significantly increased the population of STAMP2- or FSP27-positive chondrocytes as well as PKCK2-positive cells (FIG. 6C). We next examined the effect of STAMP2 overexpression on oleate-induced lipotoxicity. Overexpression of STAMP2 not only significantly prevented 1.5 mM oleate-induced cell death (FIG. 6D) but also reversed the oleate-induced decrease in the FSP27 protein expression level (FIG. 6E). Furthermore, overexpression of STAMP2 reversed the 1.5 mM oleate-induced reduction in the total LD volume (FIG. 6F).

2-7. Articular Chondrocytes Co-Incubated with Palmitate and Oleate Also Survive Through PKCK2/STAMP2/FSP27-Mediated LD Accumulation.

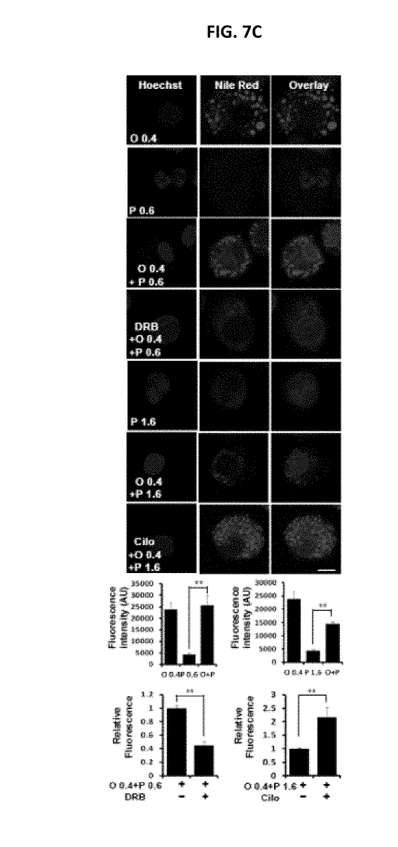

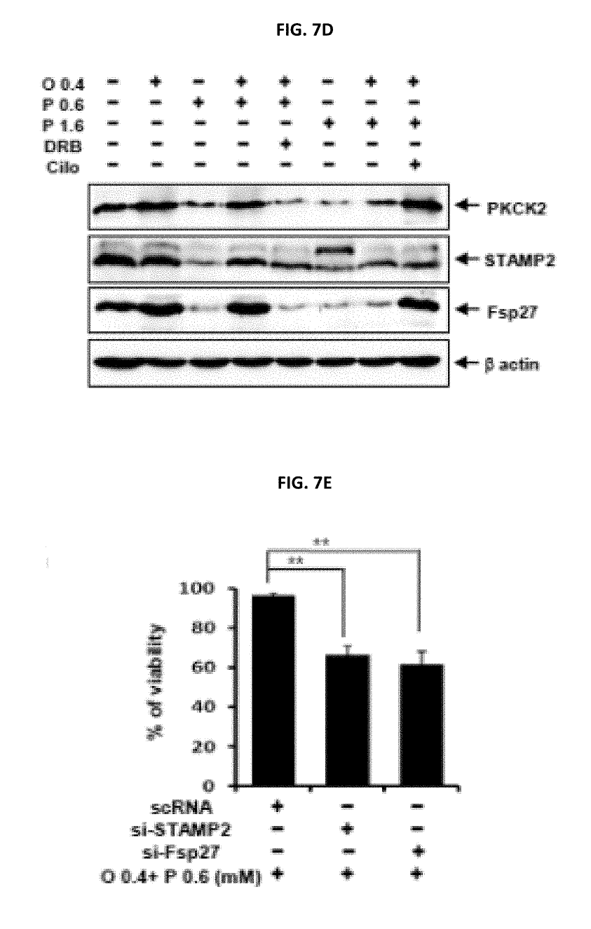

Under physiological conditions, the FFA pool contains different saturated and unsaturated species that influence each other. Palmitate and oleate are two of the most common fatty acids in articular chondrocytes. Thus, using several combinations of palmitate and oleate, we examined whether articular chondrocytes gain resistance to lipotoxicity through LD accumulation. We observed that 0.2 and 0.4 mM oleate supplementation markedly suppressed 0.4-1.8 mM palmitate-induced lipotoxicity (FIG. 7A). For further studies on LD accumulation, we chose the following two combination treatments: 0.4 mM oleate with 0.6 mM palmitate and 0.4 mM oleate with 1.6 mM palmitate. In these two combination treatments, oleate supplementation significantly reverse palmitate-induced lipotoxicity, and PKCK2 inhibition and augmentation showed reciprocal influences on lipotoxicity (FIG. 7B). In these combination treatments, oleate supplementation induced LD accumulation in articular chondrocytes treated with 0.6 mM or 1.6 mM palmitate. While DRB significantly reduced the LD accumulation in cells treated with 0.4 mM oleate and 0.6 mM palmitate, cilostazol augmented the LD accumulation in cells treated with 0.4 mM oleate and 1.6 mM palmitate (FIG. 7C). These findings support the notion that LD accumulation prevents lipotoxicity in articular chondrocytes co-incubated with palmitate and oleate. We further observed that the increase in LD accumulation in chondrocytes co-treated with oleate and palmitate also correlated with the increase in the expression level of PKCK2, STAMP2 and FSP27 (FIG. 7D). Moreover, siSTAMP2 and siFSP27 significantly decreased the viability of chondrocytes co-treated with 0.4 mM oleate and 0.6 mM palmitate (FIG. 7E). The data in FIGS. 6A to 6F and FIGS. 7A to 7E indicate that the PKCK2/STAMP2/FSP27 axis confers articular chondrocytes resistance to FFA-induced lipotoxicity.

2-8. Increase of Cytosolic FFAs is Correlated with Exertion of Lipotoxicity in Articular Chondrocytes.

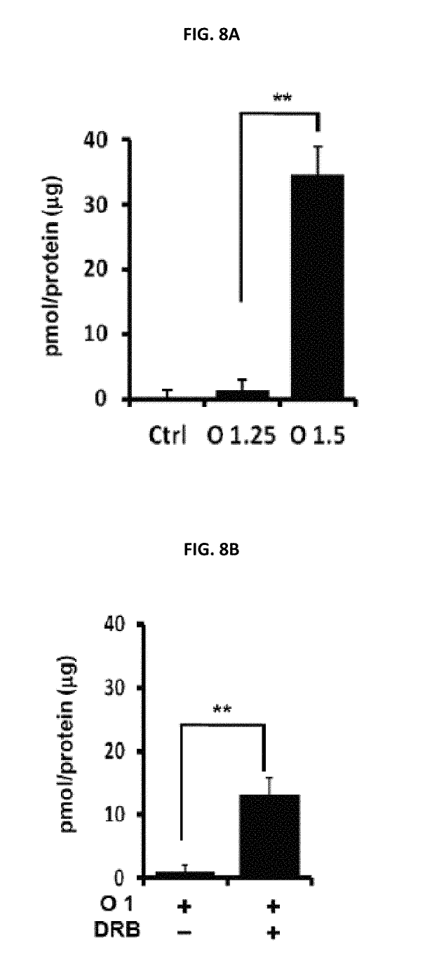

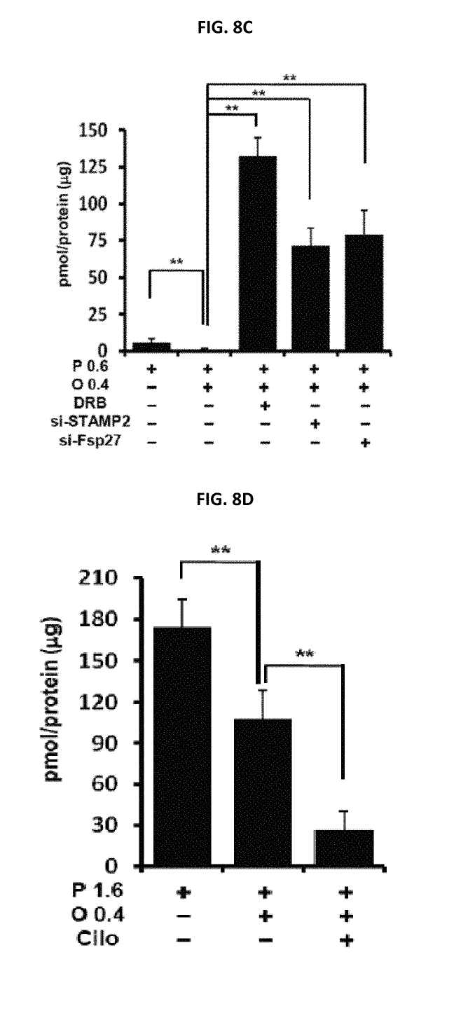

We next examined whether cytosolic FFAs, which is not incorporated in LD, is increased in articular chondrocytes which succumb to lipotoxicity. We measured total cytosolic FFA content in articular chondrocytes after various combination treatments. Notably, we observed that concentrations of cellular FFAs were significantly higher in articular chondrocytes which succumb to lipotoxicity, compared to articular chondrocytes surviving FFAs-induced lipotoxicity (FIGS. 8A to 8C). This phenotype was observed not only in articular chondrocytes treated with oleate alone (FIGS. 8A to 8B) but also in cells co-treated with palmitate and oleate (FIGS. 8C to 8D). These findings indicate that the exertion of lipotoxicity by FFAs seems to depend on the increased concentration of cellular FFAs freed from LD, supporting that articular chondrocytes survive through the sequestration of FFAs in LD.

It will be apparent to those skilled in the art that various modifications can be made to the above-described exemplary embodiments of the present invention without departing from the scope of the invention. Thus, it is intended that the present invention covers all such modifications provided they come within the scope of the appended claims and their equivalents.

What is claimed is:

1. A method for treating or preventing osteoarthritis in a subject, comprising promoting the expression of STAMP2 (Six-transmembrane protein of prostate 2) in chondrocyte in the subject.

2. The method according to claim 1, wherein the promoting the expression of STAMP2 is by administering cilostazol or TNF-α, to a subject.

3. The method according to claim 2, wherein the administration is an oral or intravenous administration.

4. The method according to claim 1, wherein the promoting the expression of STAMP2 is by administering a vehicle, into which a gene encoding STAMP2 is introduced, to a subject.

5. The method according to claim 4, wherein the vehicle is an adenovirus.

6. The method according to claim 4, wherein the administration is an intravenous administration.

7. The method of claim 1, wherein the method is for treating osteoarthritis in the subject, and the subject has osteoarthritis.

8. A method for diagnosing osteoarthritis in a subject, the method comprising:
a) measuring an expression level of STAMP2 or an amount of STAMP2 protein in chondrocyte from the subject; and
b) comparing the measured results in step a) with that in a control sample.

9. The method according to claim 8, wherein the expression level of STAMP2 is measured by any one selected from the group consisting of RT-PCR, Competitive RT-PCR, Realtime RT-PCR, RPA (RNaseprotection assay), Northern blotting and DNA chip.

10. The method according to claim 8, wherein the amount of STAMP2 protein is to measured by any one selected from the group consisting of Western blot, ELISA (enzyme linked immunosorbent assay), RIA (radioimmunoassay), radioimmunodiffusion, Ouchterlony immunodiffusion, rocket immunoelectrophoresis, IHC (immunohistochemistry), Immunoprecipitation assay, Complement fixation assay, FACS (fluorescence activated cell sorter) and protein chip.

11. A method for screening a therapeutic agent for osteoarthritis, the method comprising:
a) treating chondrocyte with a candidate agent;
b) measuring an expression level of STAMP2 or an amount of STAMP2 protein in the chondrocyte treated with the candidate agent; and
c) identifying a candidate agent as the therapeutic agent when the expression level of STAMP2 or the amount of STAMP2 protein measured in step b) is increased in the chondrocyte as compared to the chondrocyte before treatment with the candidate agent.
